(19) United States
(12) Patent Application Publication
(10) Pub. No.: US 2018/0361007 A1
(43) Pub. Date: Dec. 20, 2018

(54) ULTRAVIOLET GERMICIDAL IRRADIATION DEVICE AND METHOD

(71) Applicant: Sean Caffrey, Oakville (CA)

(72) Inventor: Sean Caffrey, Oakville (CA)

(21) Appl. No.: 15/623,132

(22) Filed: Jun. 14, 2017

(51) Int. Cl. A61L 9/20

(52) U.S. Cl. CPC A61L 9/20

(57) ABSTRACT

An ultraviolet germicidal irradiation device relating to reducing air-borne contaminants within air passing through an air-distribution duct. The device uses ultraviolet light source configured to produce germicidal UV-C light, a switchless current-conducting circuit configured to continuously conduct electrical current between a battery source and the at least one electrically-operated ultraviolet light source, and a housing to house electrically-operated ultraviolet light source and the switchless current-conducting circuit. The housing includes a mount configured to mount the housing within the air-distribution duct, a battery compartment adapted to hold the battery source, and at least one ultraviolet-light passage adapted to pass the germicidal ultraviolet light from the electrically-operated ultraviolet light source through the housing.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of disinfecting and sterilizing of existing art and more specifically relates to an apparatus for disinfecting or sterilizing with ultraviolet radiation generating means.

### RELATED ART

Over two billion dollars was spent last year in the United States alone on indoor air quality. The United States Environmental Protection Agency estimates that this industry will grow at a rate of more than ten percent a year until 2025. Studies show that indoor air may be five to fifteen times more polluted than outside air. Consumers are concerned about germs, mold, and other contaminates in their homes. A consumer product providing air-quality protection in every room at a cost effect price would benefit many.

Prior attempts have been made to address the issue of indoor air quality. For example, U.S. Pat. No. 7,300,499 to Fleisher relates to an airplane air purifier. The described airplane air purifier employs high voltage electrostatic ionic air charging grid and precipitator plates for the removal of particulates and contaminants, together with germicidal capabilities provided by an ultraviolet wavelength band UVC illuminator within the purifier. The purifier is adapted to operate from the aircraft passenger cabin electrical supply or alternately from replaceable or rechargeable batteries. The purifier provided with one or more nozzle adapters to removably and supportively install the air purifier to the typical varieties of aircraft passenger air vent nozzles. The air purifier is small in size and light in weight so as to be easily carried onboard the flight and installed without issue to the air vent nozzle, whereby the air purifier purifies the ducted cabin air in the plane before diffusing into the passenger's breathing air space. The airplane air purifier of Fleisher is both complex and incompatible with the heating, ventilation, and air conditioning (HVAC) duct system of a building structure.

### SUMMARY OF THE INVENTION

In view of the foregoing disadvantages inherent in the known an apparatus for disinfecting or sterilizing art, the present disclosure provides a novel ultraviolet germicidal irradiation device and method. The general purpose of the present disclosure, which will be described subsequently in greater detail, is to provide an ultraviolet germicidal irradiation device and method.

An ultraviolet germicidal irradiation device relating to reducing air-borne contaminants within air passing through an air-distribution duct is disclosed herein. The ultraviolet germicidal irradiation device includes at least one electrically-operated ultraviolet light source configured to produce germicidal ultraviolet light; a switchless current-conducting circuit configured to continuously conduct electrical current between a battery source and the at least one electrically-operated ultraviolet light source; and a housing to enclose the at least one electrically-operated ultraviolet light source and the switchless current-conducting circuit. The housing includes a mount configured to mount the housing within the air-distribution duct, a battery compartment adapted to hold the battery source, and at least one ultraviolet-light passage adapted to pass the germicidal ultraviolet light from the at least one electrically-operated ultraviolet light source through the housing.

Moreover, it provides such a device, wherein the at least one electrically-operated ultraviolet light source is configured to emit ultraviolet light having a wavelength of between about 400 nanometers and about 10 nanometers. More specifically, it provides such a device wherein the at least one electrically-operated ultraviolet light source emits UV-C germicidal ultraviolet light having a wavelength of between about 300 nanometers and about 100 nanometers. Also, it provides such a device wherein the at least one electrically-operated ultraviolet light source includes at least one light-emitting diode. In addition, it provides such a device wherein the housing includes at least one reflector configured to reflectively direct the germicidal ultraviolet light toward the air passing through the air-distribution duct.

In addition, the device may further comprise the battery source, wherein the battery source is configured to supply operating electrical current to the at least one electrically-operated ultraviolet light source. In one embodiment of the present disclosure, the battery source includes at least one single-use electrochemical cell. In one embodiment of the present disclosure, the battery source includes at least one rechargeable electrochemical cell.

Furthermore, it provides such a device wherein the at least one ultraviolet-light passage includes at least one light spreading optical element configured to spread the germicidal ultraviolet light emitted by the at least one electrically-operated ultraviolet light source. In one embodiment of the present disclosure, the mount includes a permanent magnet adapted to removeably engage at least one ferromagnetic surface of the air-distribution duct. In one embodiment of the present disclosure, the mount includes at least one mounting clip adapted to removably mount the housing to a supply-air register of the air-distribution duct.

In addition, it provides such a device wherein the housing includes an air-supply register configured to engage a discharge opening of the of the air-distribution duct, wherein the mount includes a flange configured to engage a discharge opening of the of the air-distribution duct, wherein the air-supply register is configured to direct the germicidal ultraviolet light toward an interior portion of the air-distribution duct. In one embodiment of the present disclosure, the air-supply register is constructed primarily of a metallic material.

Furthermore, it provides such a device wherein the housing includes an aerodynamic shape configured to promote low-drag movement of the air passing through an air-distribution duct. Even further, it provides such a device wherein the housing includes a user-operable access cover configured to provide user access to the battery compartment. Moreover, it provides such a device wherein the housing is constructed from plastic.

In addition, it provides such a device, further including set of instructions; and wherein the device is arranged as a kit. Even further, it provides such a device, wherein the set of instructions includes a service-check date reminder adapted to remind a user of a date on which the device requires a service check.

In accordance with another preferred embodiment hereof, this invention provides a method of reducing air-borne contaminants in air conducted through an air-distribution duct, the method including the steps of: providing an ultraviolet germicidal irradiation device including at least one electrically-operated ultraviolet light source configured to produce germicidal ultraviolet light; a switchless current-conducting circuit configured to continuously conduct electrical current between a battery source and the at least one electrically-operated ultraviolet light source; and a housing to house the at least one electrically-operated ultraviolet light source and the switchless current-conducting circuit, the housing including a mount configured to mount the housing within the air-distribution duct, a battery compartment adapted to hold the battery source, and at least one ultraviolet-light passage adapted to pass the germicidal ultraviolet light from the at least one electrically-operated ultraviolet light source through the housing; enabling the operation of the at least one electrically-operated ultraviolet light source by operably coupling the battery source and the switchless current-conducting circuit; and mounting the housing within the air-distribution duct.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, an ultraviolet germicidal irradiation device and method, constructed and operative according to the teachings of the present disclosure.

FIG. 1 is a front perspective view of the ultraviolet germicidal irradiation device, according to an embodiment of the present disclosure.

FIG. 2 is a perspective view of the ultraviolet germicidal irradiation device of FIG. 1 during an ‘in-use’ condition, according to an embodiment of the disclosure.

FIG. 3 is a diagrammatic sectional view of the ultraviolet germicidal irradiation device during the ‘in-use’ condition of FIG. 2, according to an embodiment of the present disclosure.

FIG. 4 is a rear perspective view of the ultraviolet germicidal irradiation device of FIG. 1, according to an embodiment of the present disclosure.

FIG. 5 is a front exploded perspective view of the ultraviolet germicidal irradiation device of FIG. 1, according to an embodiment of the present disclosure.

FIG. 6 is a rear exploded perspective view of the ultraviolet germicidal irradiation device of FIG. 1, according to an embodiment of the present disclosure.

FIG. 7 is a diagrammatic sectional view of an alternate ultraviolet germicidal irradiation device during an ‘in-use’ condition, according to another embodiment of the present disclosure.

FIG. 8 is an electrical circuit diagram, according to the embodiments of the present disclosure.

FIG. 9 is a flow diagram illustrating a method of use for the present invention, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to an apparatus for disinfecting or sterilizing and more particularly to an ultraviolet germicidal irradiation device and method as used to reduce air-borne contaminants within air passing through an indoor air-distribution duct.

Generally, the disclosed device provides a source of germicidal Ultraviolet C (UV-C) light that fits in or adjacent to the supply vent of an indoor ventilation system or connects to a supply register cover to purify air to each zone. The device kills airborne germs at the final stage of the indoor ventilation system by preventing air from picking up germs, mold, and dust as it travels through the ductwork. Versions of the device may include one or more UV-C Light Emitting Diodes (LED) and a magnet or vent-cover clip. In one version, the device is incorporated within a floor, wall, or ceiling supply register or is clipped to a vent cover at the last stage of the air traveling though the duct system. Air passing through the UV-C light is purified just prior to entering the room to prevent the air from picking up contaminates in the duct. The system may be battery operated for ease of installation and use.

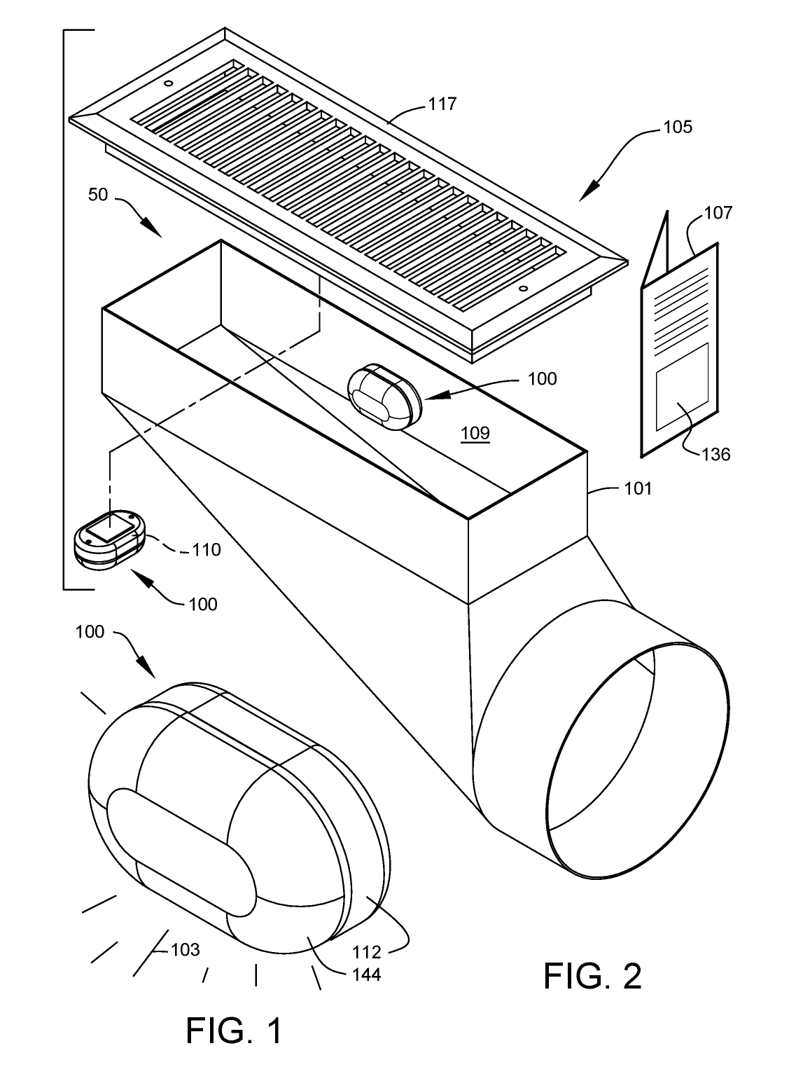

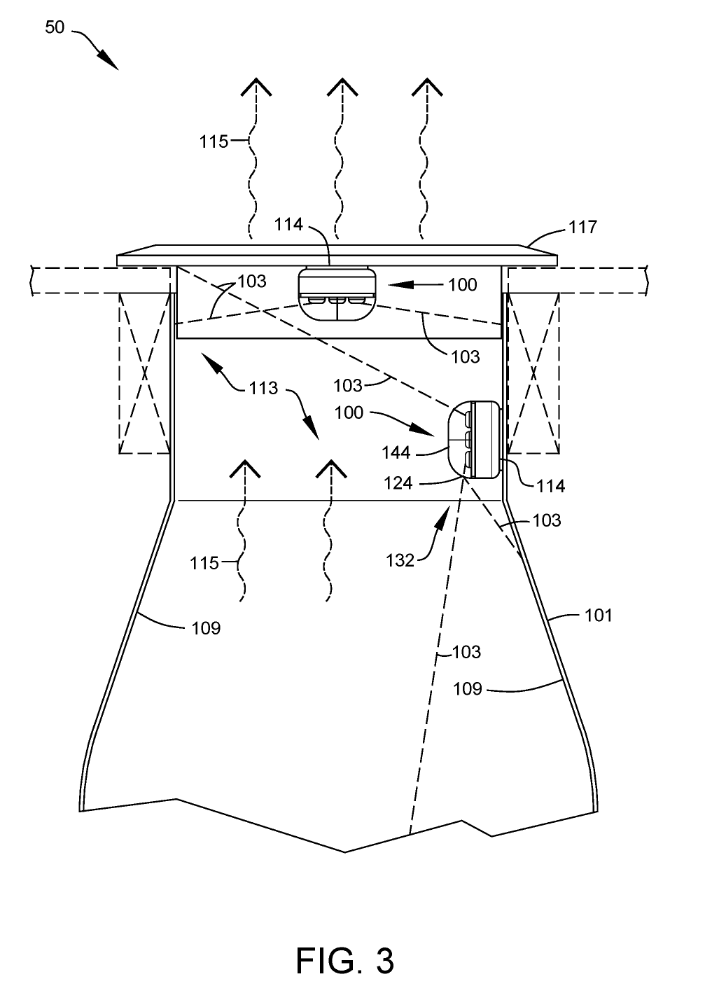

Referring now more specifically to the drawings by numerals of reference, there is shown in FIGS. 1-8, various views of an ultraviolet germicidal irradiation device 100. FIG. 1 is a front perspective view of the device 100, according to an embodiment of the present disclosure. FIG. 2 is a perspective view illustrating the device 100 during an ‘in-use’ condition, according to an embodiment of the disclosure. FIG. 3 is a diagrammatic sectional view of the device 100 during the ‘in-use’ condition of FIG. 2, according to the embodiment of FIG. 1. The illustrations of FIG. 2 and FIG. 3 show the device 100 during the ‘in-use’ condition 50, according to an embodiment of the present disclosure. Here, the device 100 may be beneficial for use to reduce air-borne contaminants within air 115 passing through an air-distribution duct 101 using germicidal ultraviolet light 103. The device 100 is configured to be mountable at a selected position within the air-distribution duct 101, as shown. More specifically, the device 100 is designed to allow a user to attach the device to a supply-air register 117 or an interior surface 109 of the air-distribution duct 101, as shown. It should be noted that the term “register”, as used in the present disclosure, will be understood to include all forms of floor, wall, and ceiling accessories employed to cover the opening of HVAC ductwork. Such registers may comprise simple fixed grills or may include moving parts, capable of directing or reducing airflow.

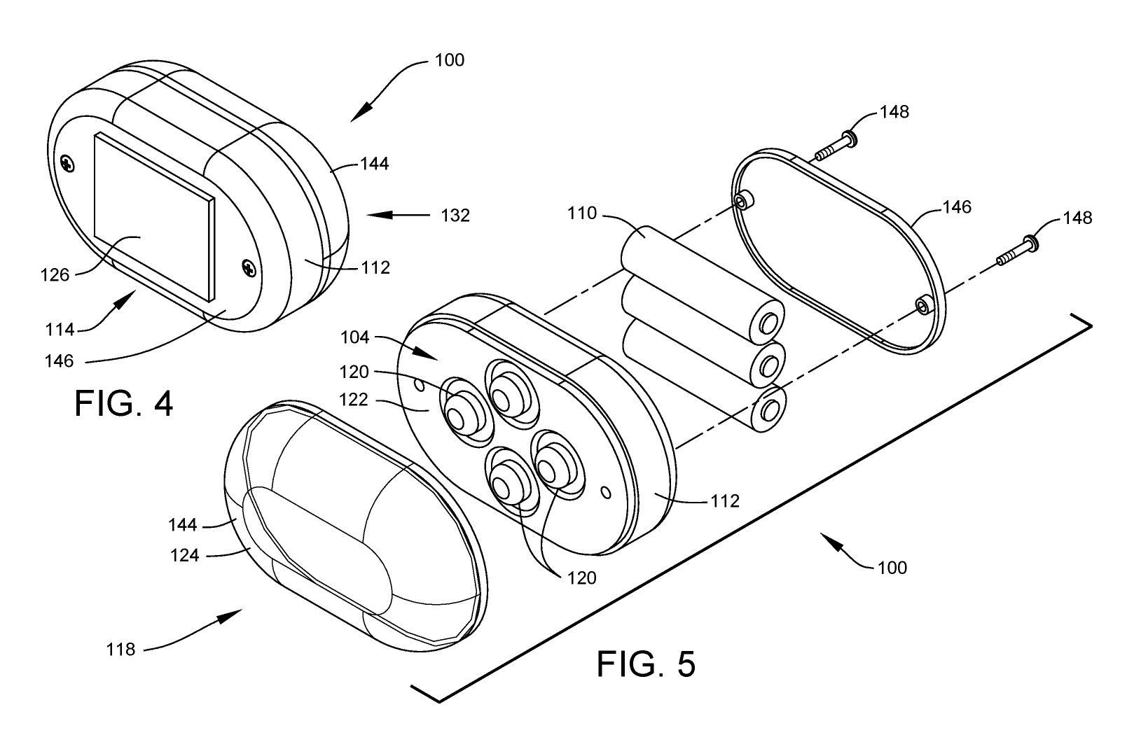

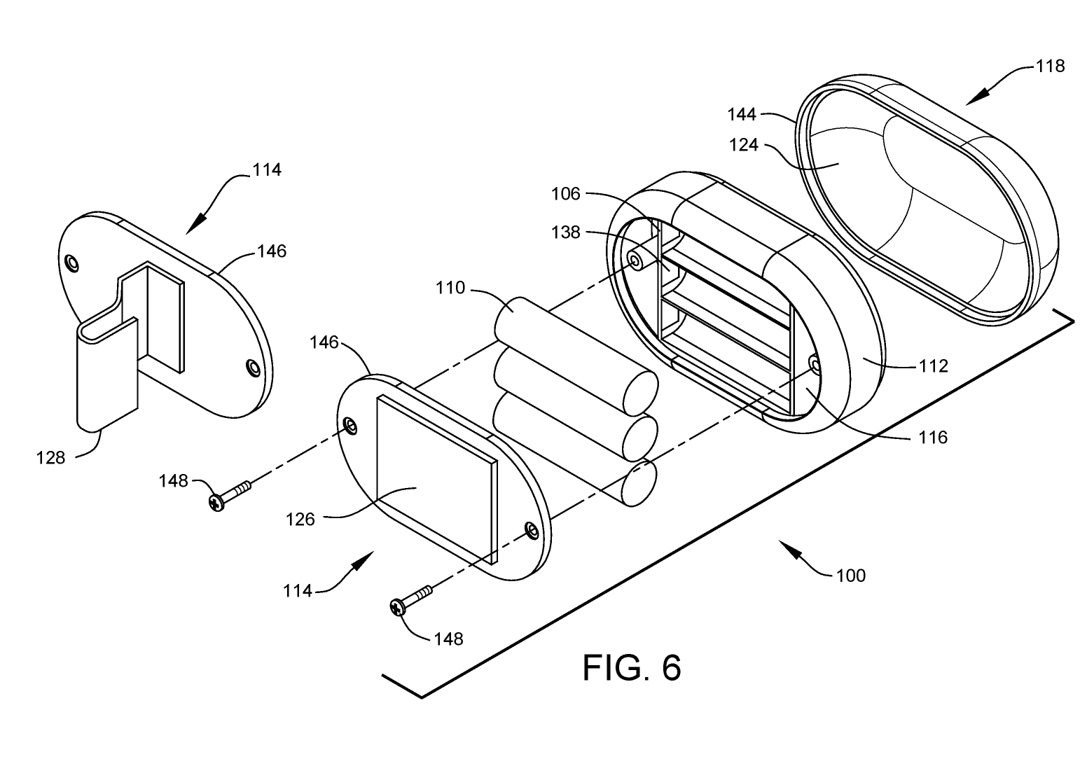

FIG. 4 is a rear perspective view of the device 100, according to the embodiment of FIG. 1. FIG. 5 is a front exploded perspective view of the device 100, according to the embodiment of FIG. 1. FIG. 6 is a rear exploded perspective view of the device 100 of FIG. 1, according to an embodiment of the present disclosure.

FIG. 1 through FIG. 6 illustrate the preferred structures and arrangements of one embodiment the device 100. As illustrated, the device 100 may include an electrically-operated ultraviolet light source 104 configured to produce germicidal ultraviolet light 103 and a switchless current-conducting circuit 106 configured to continuously conduct electricity between a direct current (DC) battery source 110 and the electrically-operated ultraviolet light source 104 (see also the diagram of FIG. 8). The electrically-operated ultraviolet light source 104 is configured to emit light in the ultraviolet (UV) spectrum. More specifically, the electrically-operated ultraviolet light source 104 may be supplied as one or more light-emitting diodes (LED) 120 emitting germicidal ultraviolet light 103 having a wavelength of between about 300 nanometers and about 100 nanometers. The circuit diagram of FIG. 8 shows one version of the switchless current-conducting circuit 106. The use of UV-C LEDs allows the device to operate with high energy efficiency. Because the LED modules consume a fraction of the power of traditional mercury-based UV-C lamps, the battery source 110 is capable of operating the device 100 for extended periods before replacement or recharging of the battery source 110 is required. FIG. 5 shows an electrically-operated ultraviolet light source 104 utilizing multiple LEDs 120. Although four LEDs 120 are shown, it is noted that the device 100 may be designed to utilize any selected number of LED modules.

For reliability, simplicity of operation, and cost, the current-conducting circuit 106 is designed to minimize the number of electronic components utilized. More specifically, the current-conducting circuit 106 has been refined to exclude all but the electric ally-operated ultraviolet light source 104, the battery source 110, and an efficient arrangement of electrical conductors 138. If needed, a current-limiting resistor R1 may be provided to limit the current passing through the parallel arrangement of LEDs 120 forming the electrically-operated ultraviolet light source 104. The circuit is designed to immediately and continuously energize the LEDs 120 on connecting the electrical conductors 138 to the battery source 110; thus, no ON/OFF switch or other current-controlling features are required. Upon reading this specification, it should be appreciated that, under appropriate circumstances, considering such issues as user preferences, design preference, electrical requirements, marketing preferences, cost, available materials, technological advances, etc., other circuit arrangements such as, for example, alternate layouts, the use of more or fewer LED modules, the inclusion of additional control components, the inclusion of battery-monitoring features, powering the device from an external electrical power source, etc., may be sufficient.

A housing 112 is provided to enclose the electrically-operated ultraviolet light source 104 and the switchless current-conducting circuit 106. The housing 112 may be constructed from a plastic or similar durable and light-weight material. The housing 112 includes at least one mount 114 allowing a user to mount the housing 112 within the air-distribution duct 101. In addition, the housing 112 may include a battery compartment 116 adapted to hold the battery source 110 and at least one ultraviolet-light passage 118 adapted to pass the germicidal ultraviolet light 103 from the ultraviolet light source 104 through the housing 112. In the depicted embodiment of FIG. 1 through FIG. 6, the ultraviolet-light passage 118 is a cover element having a clear or translucent wall 144, as shown. The clear or translucent wall 144 may include one or more light-spreading optical elements 124 designed to spread the germicidal ultraviolet light 103 emitted by the LED modules over a larger interior region 113 of the air-distribution duct 101, as best shown in FIG. 3. The optical elements 124 of the clear or translucent wall 144 may include a plurality of refractive and/or reflective facets to direct the ultraviolet light 103 toward the interior surfaces 109 of the air-distribution duct 101, as shown. In addition, the housing 112 may include at least one reflective surface 122 to further assist in directing the UV-C light toward the air 115 passing through the air-distribution duct 101.

The optical elements 124 may also be configured to limit the amount of UV-C light passing outward through the supply-air register 117. Upon reading this specification, it should be appreciated that, under appropriate circumstances, considering such issues as user preferences, design preference, LED configuration, marketing preferences, cost, available materials, technological advances, etc., other UV-light distribution arrangements such as, for example, alternate LED layouts, the use of more or fewer LEDs modules, mounting LED modules to the outside of the housing, using one or more concave lenses to modify the distribution of the UV-C light, etc., may be sufficient.

The battery source 110 of the device 100 may include a plurality of direct current (DC) electrochemical cells, as shown. For example, the device 100 may be configured to operate on three AA or AAA batteries, with each cell supplying about 1.5 volts. The battery compartment 116 of the housing 112 may be molded or otherwise formed to hold the cells in an operable position in contact with the electrical conductors 138 of the current-conducting circuit 106. The housing 112 of the device 100 may include a user-operable access cover 146 configured to provide user access to the battery compartment 116. The access cover 146 may be secured by a set of threaded fasteners 148, as shown, or by a snap-fit configuration known to those skilled in the art of product-housing design. Upon reading this specification, it should be appreciated that, under appropriate circumstances, considering such issues as user preferences, design preference, electrical requirements, marketing preferences, cost, available materials, technological advances, etc., other battery arrangements such as, for example, the use of alternate battery formats, the use of fewer or more battery cells, the inclusion of charging ports to enable the use of rechargeable (secondary) battery cells, etc., may be sufficient.

In one embodiment of the present disclosure, the battery source 110 may be included with the device 100. The battery source 110 may be supplied with distribution packaging or may be pre-installed in the housing by the manufacture. In one embodiment of the present disclosure, the battery source 110 may consist of one or more primary (single-use “disposable”) electrochemical cells. For example, the single-use electrochemical cells may be commercially-available alkaline batteries. In another embodiment of the present disclosure, the battery source 110 may be one or more secondary (rechargeable) electrochemical cells. For example, the secondary (rechargeable) electrochemical cells may be selected from commercially-available nickel cadmium (NiCd), nickel metal hydride (NiMH), lithium ion (Li-ion), lithium ion polymer (Li-ion polymer), and the like.

According to embodiments of the disclosed technology, the device 100 is freely movable among various rooms and ductwork to be sterilized. The device 100 can be easily moved using the provided mount 114 of the housing 112. In one implementation of the present disclosure, the mount 114 may be a permanent magnet 126 adapted to removeably engage at least one ferromagnetic (iron-containing) surface of the air-distribution duct 101. FIG. 3 shows two preferred mounting positions of device 100. Namely, the device 100 may be magnetically mounted to the underside of the metal supply-air register 117 or to the interior surface 109 of a metal air-distribution duct 101. In another embodiment of the present disclosure, the mount 114 includes at least one mounting clip 128 adapted to removably mount the housing to the supply-air register 117 of the air-distribution duct 101, as shown. The housing 112 may include an aerodynamic shape 132 configured to promote low-drag movement of the air 115 passing through the air-distribution duct 101. It is noted that multiple devices 100 may be installed in larger HVAC duct systems.

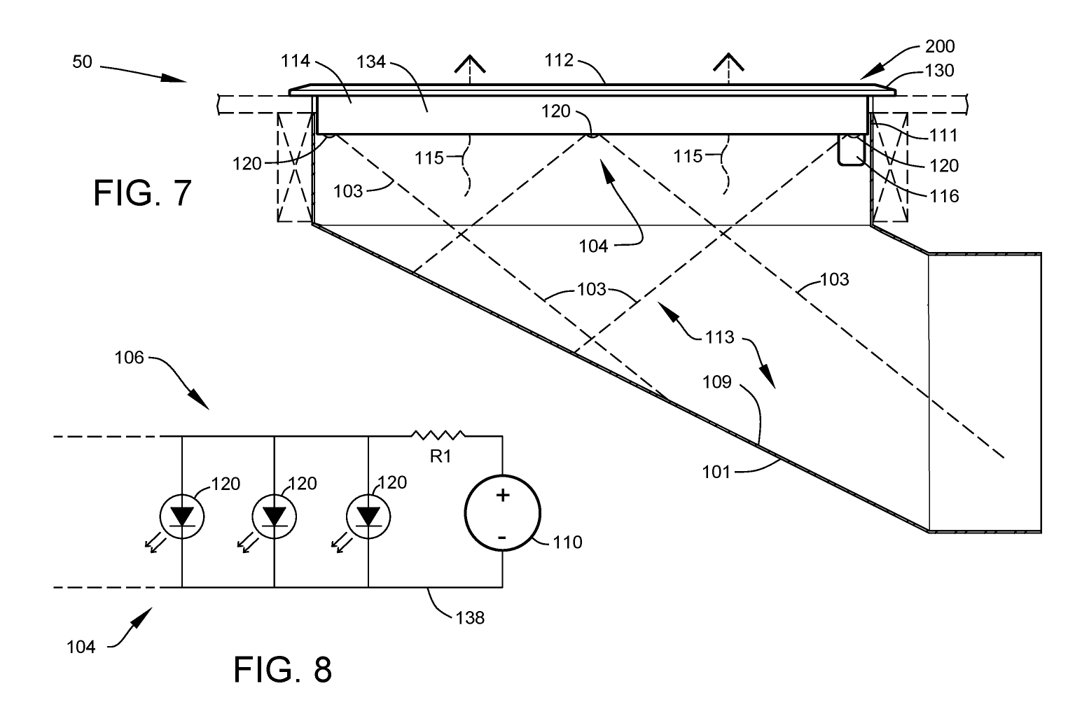

FIG. 7 is a diagrammatic sectional view of an alternate ultraviolet germicidal irradiation device 200 during an ‘in-use’ condition 50, according to another embodiment of the present disclosure. The housing 112 of the alternate ultraviolet germicidal irradiation device 200 is integrated within an air-supply register 130, as shown. The air-supply register 130 is configured to engage a discharge opening 111 of the of the air-distribution duct 101, as shown. The mount 114 includes a flange 134 configured to engage a discharge opening of the of the air-distribution duct 101 in place of a standard grill or register. The air-supply register 130 may include the above-described features of the prior embodiments, including the electrically-operated ultraviolet light source 104, current-conducting circuit 106 (hidden in the view), battery compartment 116, etc. The alternate ultraviolet germicidal irradiation device 200 is configured to direct the germicidal ultraviolet light 103 toward the interior region 113 of the air-distribution duct 101, as shown. For durability, the supply-air register 117 may be constructed mainly of a metallic material.

Either of the above-described devices may be arranged as a kit 105. In particular, the device may further include a set of instructions 107, as shown in FIG. 2. The instructions 107 may detail functional relationships in relation to the structure of the device 100 such that the device 100 can be used, maintained, or the like, in a preferred manner. In one embodiment of the kit 105, the set of instructions may include, for example, a service-check reminder 136 adapted to remind a user of a date on which the device requires a service check. Such a service-check reminder 136 may be supplied as a label on which a battery replacement date may be written. In another embodiment of present disclosure, the kit 105 may include the battery source 110. Upon reading this specification, it should be appreciated that, under appropriate circumstances, considering such issues as user preferences, design preference, structural requirements, marketing preferences, cost, available materials, technological advances, etc., other kit arrangements such as, for example, including a website link with user-instruction videos, including gaskets or sealants to assist the user during installation, including mounting assemblies adapted to engage a specific register brand or model, including tools to assist the user during installation, etc., may be sufficient.

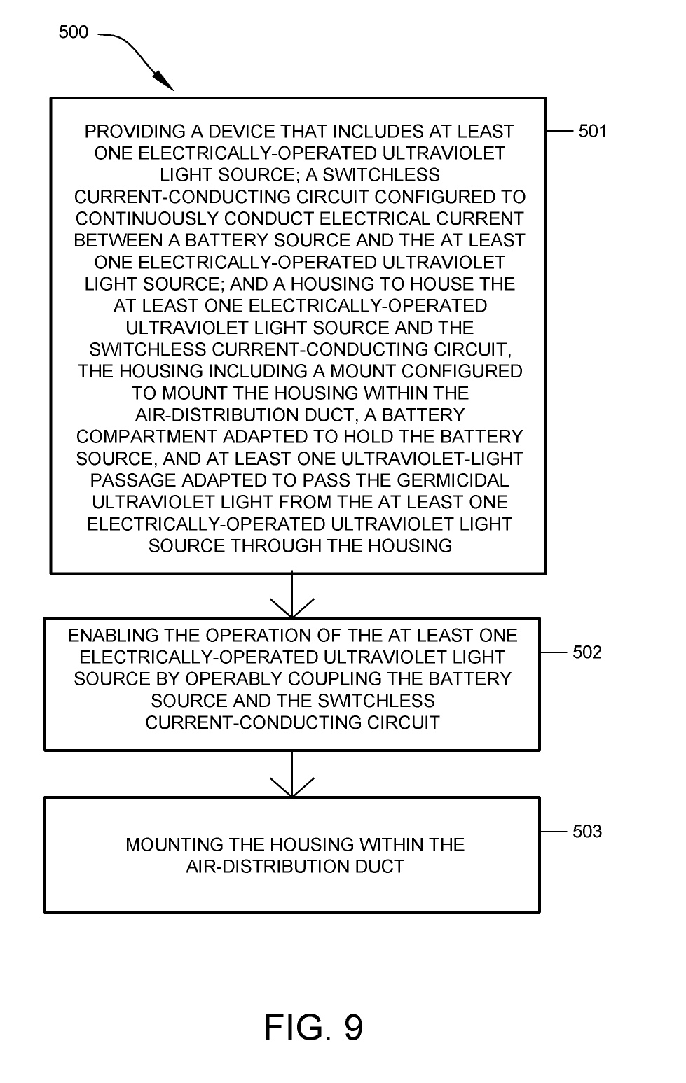

FIG. 9 is a flow diagram illustrating a method 500 for reducing air-borne contaminants within air passing through an indoor air-distribution duct. Method 500 may include one or more components or features of the above-described devices. As illustrated, the method 500 may include the steps of: step one 501, providing a device that includes at least one electrically-operated ultraviolet light source configured to produce germicidal ultraviolet light; a switchless current-conducting circuit configured to continuously conduct electrical current between a battery source and the at least one electrically-operated ultraviolet light source; and a housing to house the at least one electrically-operated ultraviolet light source and the switchless current-conducting circuit, the housing including a mount configured to mount the housing within the air-distribution duct, a battery compartment adapted to hold the battery source, and at least one ultraviolet-light passage adapted to pass the germicidal ultraviolet light from the at least one electrically-operated ultraviolet light source through the housing; step two 502, enabling the operation of the at least one electrically-operated ultraviolet light source by operably coupling the battery source and the switchless current-conducting circuit; and step three 503, mounting the housing within the air-distribution duct.

It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for reducing air-borne contaminants within air passing through an indoor air-distribution duct are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An ultraviolet germicidal irradiation device relating to reducing air-borne contaminants within air passing through an air-distribution duct, the device comprising:
at least one electrically-operated ultraviolet light source configured to produce germicidal ultraviolet light;
a switchless current-conducting circuit configured to continuously conduct electrical current between a battery source and the at least one electrically-operated ultraviolet light source; and
a housing to house the at least one electrically-operated ultraviolet light source and the switchless current-conducting circuit, the housing comprising a mount configured to mount the housing within the air-distribution duct, a battery compartment adapted to hold the battery source, and at least one ultraviolet-light passage adapted to pass the germicidal ultraviolet light from the at least one electrically-operated ultraviolet light source through the housing.

2. The ultraviolet germicidal irradiation device of claim 1, wherein the at least one electrically-operated ultraviolet light source is configured to emit the germicidal ultraviolet light in wavelengths between about 400 nanometers and about 10 nanometers.

3. The ultraviolet germicidal irradiation device of claim 1, wherein the at least one electrically-operated ultraviolet light source is configured to emit the germicidal ultraviolet light in wavelengths between about 300 nanometers and about 100 nanometers.

4. The ultraviolet germicidal irradiation device of claim 1, wherein the at least one electrically-operated ultraviolet light source comprises at least one light-emitting diode.

5. The ultraviolet germicidal irradiation device of claim 1, wherein the housing comprises at least one reflector configured to reflectively direct the germicidal ultraviolet light toward the air passing through the air-distribution duct.

6. The ultraviolet germicidal irradiation device of claim 1, further comprising the battery source, wherein the battery source is configured to supply operating electrical current to the at least one electrically-operated ultraviolet light source.

7. The ultraviolet germicidal irradiation device of claim 6, wherein the battery source comprises at least one single-use electrochemical cell.

8. The ultraviolet germicidal irradiation device of claim 6, wherein the battery source comprises at least one rechargeable electrochemical cell.

9. The ultraviolet germicidal irradiation device of claim 1, wherein the at least one ultraviolet-light passage comprises at least one light spreading optical element configured to spread the germicidal ultraviolet light emitted by the at least one electrically-operated ultraviolet light source.

10. The ultraviolet germicidal irradiation device of claim 1, wherein the mount comprises permanent magnet adapted to removeably engage at least one ferromagnetic surface of the air-distribution duct.

11. The ultraviolet germicidal irradiation device of claim 1, wherein the wherein the mount comprises at least one mounting clip adapted to removably mount the housing to an air-supply register of the air-distribution duct.

12. The ultraviolet germicidal irradiation device of claim 1, wherein
the housing comprises an air-supply register configured to engage a discharge opening of the of the air-distribution duct;
the mount comprises a flange configured to engage a discharge opening of the of the air-distribution duct; and
the air-supply register is configured to direct the germicidal ultraviolet light toward an interior portion of the air-distribution duct.

13. The ultraviolet germicidal irradiation device of claim 9, wherein the air-supply register is constructed primarily of a metallic material.

14. The ultraviolet germicidal irradiation device of claim 1, wherein the housing comprises an aerodynamic shape configured to promote low-drag movement of the air passing through an air-distribution duct.

15. The ultraviolet germicidal irradiation device of claim 1, wherein the housing comprises a user-operable access cover configured to provide user access to the battery compartment.

16. The ultraviolet germicidal irradiation device of claim 1, wherein the housing is constructed from plastic.

17. An ultraviolet germicidal irradiation device relating to reducing air-borne contaminants within air passing through an air-distribution duct, the device comprising:
at least one electrically-operated ultraviolet light source configured to produce germicidal ultraviolet light;
a switchless current-conducting circuit configured to continuously conduct electrical current between a battery source and the at least one electrically-operated ultraviolet light source; and
a housing to house the at least one electrically-operated ultraviolet light source and the switchless current-conducting circuit;
wherein the housing comprises
a mount configured to mount the housing within the air-distribution duct,
a battery compartment adapted to hold the battery source, and
at least one ultraviolet-light passage adapted to pass the germicidal ultraviolet light from the at least one electrically-operated ultraviolet light source through the housing;
wherein the at least one electrically-operated ultraviolet light source emits the germicidal ultraviolet light in wavelengths between about 300 nanometers and about 100 nanometers;
wherein the at least one electrically-operated ultraviolet light source comprises at least one light-emitting diode; and
wherein the housing comprises a user-operable access cover configured to provide user access to the battery compartment.

18. The ultraviolet germicidal irradiation device of claim 17, wherein
the housing comprises an air-supply register configured to engage a discharge opening of the of the air-distribution duct; and
the air-supply register is configured to direct the germicidal ultraviolet light toward an interior portion of the air-distribution duct.

19. The ultraviolet germicidal irradiation device of claim 17, further comprising set of instructions; and
wherein the device is arranged as a kit.

20. A method of reducing air-borne contaminants in air conducted through an air-distribution duct, the method comprising the steps of:
providing an ultraviolet germicidal irradiation device comprising
at east one electrically-operated ultraviolet light source configured to produce germicidal ultraviolet light;
a switchless current-conducting circuit configured to continuously conduct electrical current between a battery source and the at least one electrically-operated ultraviolet light source; and
a housing to house the at least one electrically-operated ultraviolet light source and the switchless current-conducting circuit, the housing comprising a mount configured to mount the housing within the air-distribution duct, a battery compartment adapted to hold the battery source, and at least one ultraviolet-light passage adapted to pass the germicidal ultraviolet light from the at least one electrically-operated ultraviolet light source through the housing;
enabling the operation of the at least one electrically-operated ultraviolet light source by operably coupling the battery source and the switchless current-conducting circuit; and
mounting the housing within the air-distribution duct.
